(19) United States
(12) Patent Application Publication
(10) Pub. No.: US 2025/0295583 A1
(43) Pub. Date: Sep. 25, 2025

(54) INHALED HEDGEHOG INHIBITORS

(71) Applicant: WBX PHARMA, INC., Medina, WA (US)

(72) Inventors: Alan Bruce Montgomery, Medina, WA (US); Kelly Lee Otto, Seattle, WA (US)

(21) Appl. No.: 19/088,764

(22) Filed: Mar. 24, 2025

Related U.S. Application Data
(60) Provisional application No. 63/568,592, filed on Mar. 22, 2024.

(51) Int. Cl. A61K 9/00; A61K 9/14; A61K 31/4402; A61K 31/502; A61P 11/00

(52) U.S. Cl. CPC A61K 9/0075; A61K 9/145; A61K 31/4402; A61K 31/502; A61P 11/00

(57) ABSTRACT

The invention is hedgehog inhibitors delivered by inhalation to treat pulmonary fibrosis. The invention includes compositions, formulations, methods of treatment, treatment scheduling, combinations with chemotherapeutic agents, and methods of manufacturing a dry powder hedgehog inhibitor formulation design to enable deposition in the deep lung based on achieving therapeutically effective formulation specifics and particle size distribution. In specific examples, vismodegib or taladegib is formulated with an excipient, such as mannitol, trehalose, or L-leucine terrific necessary parameters for advantageous delivery to a patient. The formulations are designed to be placed in a device for dry powder inhalation that is cooperatively designed and configured to deliver the dose resulting in a reduction in the progression of a progressive pulmonary fibrotic disorder.

### BACKGROUND OF THE INVENTION

Pulmonary fibrosis (PF) is a group of progressive fatal diseases that lead to inflammation and/or scarring of the lung resulting in reduced ability to absorb oxygen. The mechanism involves repeated injury to the alveolar epithelium, abnormal wound healing, and excessive deposition of extracellular matrix, particularly collagen, in the lung interstitium. Key contributing factors include chronic inflammation, fibroblast activation, epithelial-to-mesenchymal transition (EMT), and dysregulation of growth factors such as transforming growth factor-beta (TGF-β). Hypoxia, oxidative stress, and genetic predisposition may further influence disease. Pulmonary fibrosis afflicts more than 250,000 Americans. The most common symptoms of PF are a persistent cough, shortness of breath and fatigue. In some patients, lung scarring (fibrosis) is clearly linked to another illness or results from medication side effects, chest radiation treatment, environmental or occupational exposures known to cause pulmonary fibrosis. When the fibrosis continues to progress despite management of the underlying cause (e.g., immunomodulation for connective tissue disease), they are referred to as “progressive disease despite management”. When the cause is not identified, the disease is referred to as “idiopathic”. Although various forms of fibrotic lung diseases have an unknown cause, the disease known as idiopathic pulmonary fibrosis (IPF) is the most prevalent. For PF with known causes, there are five main categories: Drug-induced, Radiation-induced, Environmental, Autoimmune, and Occupational. Regardless of initial cause of pulmonary fibrosis, the pathophysiology of all causes is similar.

Pulmonary fibrosis manifests as progressive fibrosis in pulmonary alveolus interstitium and decreases respiratory function due to continuous excessive extracellular matrix component induction derived from the dysfunction of alveolar epithelial cells. Median survival after initiation of anti-fibrotic therapy is about 2.5 years (Dempsey) Steroids and immunosuppressant agents are not effective for IPF or in patients with progressive PF despite management. Currently, anti-fibrosis agents are used in the clinical management of these diseases, however, the efficacy is limited, and serios adverse effects (SAEs) are well documented.

IPF and PPF statistics for survival are well known. Median survival is about 3 years Platenburg M G J P, van der Vis J J, Grutters J C, van Moorsel C H M. Decreased Survival and Lung Function in Progressive Pulmonary Fibrosis. Medicina (Kaunas). 2023 Feb. 5; 59(2):296. doi: 10.3390/medicina59020296. PMID: 36837496; PMC/D: PMC9962949. Dempsey T M, Payne S, Sangaralingham L, Yao X, Shah N D, Limper A H. Adoption of the antifibrotic medications pirfenidone and nintedanib for patients with idiopathic pulmonary fibrosis. Annals of the American Thoracic Society. 2021 July; 18(7):1121-8. Rajan S K, Cottin V, Dhar R, Danoff S, Flaherty K R, Brown K K, Mohan A, Renzoni E, Mohan M, Udwadia Z, Shenoy P, Currow D, Devraj A, Jankharia B, Kulshrestha R, Jones S, Ravaglia C, Quadrelli S, Iyer R, Dhooria S, Kolb M, Wells A U. Progressive pulmonary fibrosis: an expert group consensus statement. Eur Respir J. 2023 Mar. 30; 61(3):2103187. doi: 10.1183/13993003.03187-2021. PMID: 36517177; PMCID: PMC10060665. The prognosis from a primary diagnosis of progressive pulmonary fibrosis is poor and can progress at varying speeds but is uniformly fatal unless another morbid condition occurs first. Kirkkainen M, Nurmi H, Kettunen H P, Selander T, Purokivi M, Kaarteenaho R. Underlying and immediate causes of death in patients with idiopathic pulmonary fibrosis. BMC Pulmonary Medicine. 2018 Dec. 18: 1-0.

Three principal treatment options exist 1) lung transplantation which has a median survival of 5 years mostly due to chronic rejection of the transplant. Only 5,000 lung transplants are done per year in the United States, and the total surviving population for lung transplant recipients is around 20,000 and so lung transplant is not a complete solution for the vast majority of patients. 2) oral drugs, pirfenidone (Esbriet®); and 3) nintedanib (Ofev®) are FDA approved for the treatment of IPF, the latter is also approved for PPF. Neither drug given orally alone as a monotherapy is curative; both slow the progression of the disease by about 50%. These oral therapies are associated with significant adverse effects, including gastrointestinal intolerance, liver toxicity, and other systemic side effects. As a result, patient compliance is a major issue, with only 25% of eligible patients initiating therapy and many discontinuing treatments within 300 days due to intolerability. The incidence and nature of adverse events associated with both drugs is extremely important in the older patient population who frequently have co-morbidities: only 25% of patients ever to receive either drug and for those who do initiate treatment, will similarly discontinue treatment by 300 days, so the discovery of a more effective and tolerable therapy is vital.

Despite the availability of lung transplantation and oral antifibrotic medications, an urgent need remains for alternative therapeutic approaches that can effectively target pulmonary fibrosis while minimizing systemic toxicity and improving patient compliance and the current standard of care has notable drawbacks and ineffectiveness and practical tolerability due to the high discontinuation rates associated with oral antifibrotic therapies. Accordingly, an urgent need for improved therapeutic strategies that enhance the efficacy of existing drugs while reducing systemic adverse effects, thereby providing a more tolerable and effective treatment option in a patient population having few viable options and a poor long-term prognosis.

The foregoing background description includes information that may be useful in understanding the present disclosure. It is not admission that any of the information provided herein is prior art or relevant to the presently claimed method and composition thereof, or that any publication specifically or implicitly referenced is prior art.

### BRIEF SUMMARY OF THE INVENTION

The present invention encompasses formulations, delivery methods, and devices and systems to enable the delivery of inhaled hedgehog pathway inhibitors using dry powder inhalation (DPI) systems. Specifically, the present disclosure focuses on optimizing a number of chemical and physical characteristics of a dry powder hedgehog inhibitor including parameters such as particle size distribution needed to achieve targeted deep lung deposition, chemical and physical parameters maximizing therapeutic efficacy while minimizing systemic side effects, and treatment methodologies enabling the use of these modalities to achieve an improved treatment option for PF regardless of the initial cause

The present invention also includes methods for manufacturing an inhaled dry powder formulation of a hedgehog pathway inhibitor capable of delivering the clinical benefits described herein. The manufacturing method comprises a step of dissolving one of the known hedgehog inhibitors, such as vismodegib or taladegib in a solvent, for example, comprising ethanol and water in a fixed and predetermined ratio. The method further comprises a step of adding an excipient, in the examples below and as shown in the Figures selected from a group consisting of mannitol, trehalose, and L-leucine and combinations thereof and other excipients featuring common physiological parameters and the ability to form the particular particle forms disclosed here. The method further comprises a step of spray drying the mixture to form a dry powder of a combination of the excipient and the hedgehog pathway inhibitor compound to achieve a formulation with collapsed sphere morphology for an improved aerosol performance. The method further comprises a step of encapsulating the dried powder formulation into a capsule for administration via a dry powder inhaler device to yield a drug device combination that can be provided to a progressive pulmonary fibrosis patient.

The formulations described herein enable an improved treatment modality and within the range of formulations described may exhibit a potency range (amount API preserved in drug product) between 98% to 102%. In some embodiments of the present disclosure, the dried powder formulation comprising a median particle size that facilitates deep lung deposition of the dried powder formulation and thereby ensuring effective delivery of the target hedgehog inhibitor such as vismodegib or taladegib. For example, a dry powder composition comprising an inhaled formulation of vismodegib or taladegib with a selected carrier or excipient. The dry powder formulation is prepared by spray drying a solution comprising vismodegib or taladegib in 50:50 ethanol-water mixture and an excipient selected from a group consisting of mannitol, trehalose, and L-leucine. The L-leucine content in the dry powder formulation is between a range of about 10% to about 50% and most preferably between approximately 10% to about approximately 26% by weight and equivalent surrounding values.

The dry powder formulation is contained within a dry powder inhaler capsule preferably with a unit dose of 400 micrograms (pg) to 1.2 milligram (mg) of vismodegib and could be delivered in multiple doses per day but is preferably given into doses or less. The dry powder formulation exhibits an emitted fraction efficiency of at least 84% and a fine particle distribution that ensures deposition of the dry powder formulation in the deep lung.

In another aspect, a method for treating pulmonary fibroses in a subject in need thereof comprises a step of administering an inhaled dry powder formulation of a hedgehog pathway inhibitor of vismodegib or taladegib. The patient is administered dry powder formulation from a drug delivery device is described below and the delivery device is configured to contain the API and excipient and stored to preserve the activity of the therapeutic formulation prepared by spray drying a solution the vismodegib or the taladegib compound in 50:50 ethanol-water mixture together with an excipient selected from a group consisting of mannitol, trehalose, and L-leucine and disposed in the device in a defined concentration in dosages described herein. The inhaled hedgehog pathway inhibitor achieves cellular adsorption of the API in a patient having histological characteristics of the pulmonary progressive fibrotic disorder.

The dry powder inhaler device is any device that delivers the particle population in an effective particle size range and dosage to the deep lung including a Berry RS01 device, or an equivalent dry powder inhaler including the class of high-resistance inhalers having equivalent characteristics and capable of analogous delivery parameters to achieve cellular adsorption in the deep lung.

By comparison with an oral dose, the vismodegib formulation achieves an epithelial lining fluid (ELF) concentration at least 16 times higher than the concentration achieved by equivalent 150 milligram (mg) oral dose of vismodegib.

The inhaled dry powder formulation of taladegib achieves an epithelial lining fluid (ELF) concentration of at least 29 times higher than the concentration achieved by an equivalent 200 milligram (mg) oral dose of taladegib. The dry powder formulation is administered at a dose between 400 micrograms (pg) to 1.2 milligram (mg) of vismodegib as a single capsule or up to 2.4 mg as two consecutive capsule inhalations or as a single or 2 consecutive capsule inhalations of taladegib between 200 μg to 3.6 mg.

As mentioned there remains a need for improved therapeutic strategy that improves upon the efficacy of existing drugs while reducing systemic adverse effects. Accordingly, the present disclosure provides a method for manufacturing an inhaled dry powder formulation of a hedgehog pathway inhibitor, a composition comprising an inhaled dry powder formulation of one of vismodegib or taladegib, and a method for treating pulmonary fibroses in a subject in need thereof.

The invention also includes coadministration of a hedgehog pathway inhibitor with the chemotherapeutic agent to enhance the effectiveness of each for example by co-solubilizing varying dosages of the chemotherapeutic agent, e.g., vincristine, to facilitate a dose de-escalation therapeutic schedule. Such a schedule increases the efficacy and reduces the toxicity of the chemotherapeutic agent to the patient. The hedgehog pathway modulator of the instant disclosure may additionally or alternatively be provided in a highly-concentrated injectable format for use in the instant methods of treatment. Hedgehog inhibitor formulations may be used in combination with a chemotherapeutic agent, for example by co-solubilizing varying dosages of the chemotherapeutic agent, e.g., vincristine, to facilitate a dose de-escalation therapeutic schedule. Such a schedule increases the efficacy and reduces the toxicity of the chemotherapeutic agent to the patient and reduces the doses of each drug necessary to provide a therapeutic effect thus circumventing the toxicities associated with the use of the two drugs in a conventional solo format.

The invention also includes coadministration of a hedgehog inhibitor with one of a chemotherapeutic, antifibrotic, Beta-2 agonist or co-administered with radiation therapy to enhance the effectiveness of each. For a companion to a chemotherapeutic treatment, an example is co-solubilizing varying dosages of the chemotherapeutic agent, e.g., vincristine, to facilitate a dose de-escalation therapeutic schedule by also administering a hedgehog pathway inhibitor as disclosed herein. Such a schedule increases the efficacy and reduces the toxicity of the chemotherapeutic agent to the patient. This would be advantageous in the treatment of radiation pneumonitis as the hedgehog inhibitor may also have an anti-tumor effect. Combinations with known antifibrotics such as nintedanib or pirfenidone may provide synergistic efficacy. Combinations with a Beta 2 agonist such as albuterol may open distal airways delivering more hedgehog inhibitor to the alveolar space, thus improving efficacy. Co-administration with radiation therapy has been shown to radio-sensitize some tumors providing improved efficacy.

The foregoing brief description is a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the following detailed description.

### BRIEF DESCRIPTION OF THE FIGURES

FIG. 1 is a microscopic view of 4 artifacts of successful particle engineering for 2 different leucine concentrations together with Mannitol and Trehalose. Panels B and C achieve the desired property of collapsed spheres indicating leucine surface enrichment.

FIG. 2 is a geometrical particle size distribution (GPSD) achieved by the formulations described herein in a preferred range of values validating the manufacturing process parameters and showing a low variable range maintaining particle size distribution in a desired respirable range for deep lung deposition.

FIG. 3 is fast screening impactor results for 4 separate engineered dry powder compositions for combinations of hedgehog inhibitors incorporated into the engineered dry particles and containing combinations of leucine, and alternatively mannitol and trehalose

### DETAILED DESCRIPTION OF THE INVENTION

Pulmonary fibrosis is a chronic lung condition characterized by excessive scar tissue (fibrosis) in the lungs, due to an imbalance between tissue repair and injury. The hallmark is overproduction of extracellular matrix (ECM) (collagens, fibronectin, etc.) in the lung interstitium, leading to architectural distortion of alveoli and progressive loss of lung function. Idiopathic Pulmonary Fibrosis (IPF) is the classic example, with a notoriously poor prognosis having a median survival time of only approximately 3 years following diagnosis.

The pathogenic mechanism involves a complex interplay of epithelial cell injury, abnormal wound healing with fibroblast activation, chronic inflammation, and molecular signaling loops that perpetuate fibrosis. Below is a structured overview of key processes and pathways, including the roles of inflammation, fibroblasts, epithelial-to-mesenchymal transition (EMT), ECM deposition, and mediators like TGF-β. Genetic predispositions and environmental factors (e.g. smoking, dust exposure) also influence disease development and progression.

Pulmonary fibrosis is often initiated mechanistically with repetitive micro-injury to the alveolar epithelium (especially the delicate type I pneumocytes that line the gas-exchange surface). These injuries can be caused by environmental factors (e.g. inhaled toxins, cigarette smoke, viral infection) or occur idiopathically. The injured alveolar cells undergo apoptosis or dysfunction, and denudation of the alveolar basement membrane occurs. A typical finding in IPF lung tissue is the loss of normal type I alveolar cells, with hyperplastic cuboidal type II pneumocytes attempting to regenerate the epithelium.

This indicates failed re-epithelialization and an abnormal repair response. The epithelial injury also exposes the underlying basement membrane, leading to leakage of plasma proteins like fibrin into the interstitium and activation of coagulation cascades as part of the wound response.

Early in the process, the injury triggers an acute and prominent inflammatory response in the lung that is characteristic of the progressive nature of the disease and exacerbates the long-term injurious effects of the disease. Histology often shows interstitial infiltrates of immune cells—including lymphocytes, alveolar macrophages, neutrophils, and eosinophils—in affected areas.

These inflammatory cells release cytokines (such as interleukins, TNF-α) and growth factors that initially help clear damage but also set the stage for fibrogenesis. Macrophages play a dual role: they remove debris but also secrete pro-fibrotic mediators (e.g. TGF-β1) that activate fibroblasts. Over time, a chronic, low-grade inflammation may persist in the interstitium, providing a continued source of cytokines and chemokines. However, some evidence questions the primary role of inflammation per se as the primary driver of all forms or sources of IPF and anti-inflammatories have mixed impacts on disease progression. A focus remains on wound healing where the fibrotic process becomes self-sustaining even as the initial inflammatory response subsides.

Pulmonary fibrosis prominently features a process where the alveolar epithelium becomes dysfunctional and fails to heal properly after injury. Repeated injuries and an aging epithelium (with cumulative telomere shortening) can lead to an “activated” alveolar epithelium that behaves abnormally even without ongoing insult. The remaining type II pneumocytes proliferate to cover denuded areas but often exhibit abnormal behavior: they secrete pro-fibrotic mediators, adopt altered phenotypes, or undergo apoptosis instead of maturing into type I cells. Stressed alveolar epithelial cells show signs of endoplasmic reticulum (ER) stress and unfolded protein response (UPR) activation, as seen in some familial cases with surfactant protein mutations that cause misfolded proteins. The damaged epithelium also communicates aberrantly with neighboring cells, releasing growth factors, chemokines, and developmental signaling proteins (like Wnt) that influence the surrounding tissue.

Over time, epithelial dysfunction recruits fibroblasts and inflammatory cells that drive the progressive nature of the fibrosis and driving forward the observed fibrosis. In the lungs, Transforming Growth Factor-β1 (TGF-β1) is a potent inducer of epithelial to mesenchymal transition (EMT) by triggering signaling cascades (SMAD-dependent and others) that repress epithelial markers (like E-cadherin) and induce mesenchymal markers (like vimentin and α-smooth muscle actin).

TGF-β1 is the major driver of myofibroblast differentiation, typically by engaging SMAD3 signaling in fibroblasts. Fibroblast foci are important in progressive pulmonary fibrosis because the fiber blasts produce considered the leading edge of active fibrosis, where fibroblasts are laying down new collagen and fibronectin leading to the progressive lung injury. Over time, myofibroblasts can also appear in the walls of small airways and vessels, contributing to obliteration and distortion of normal lung architecture and activated fibroblasts in turn release various growth factors (including TGF-β1) that can recruit and activate more fibroblasts, creating a positive feedback loop that upsets the normal, healthy balance between tissue deposition and degradation and skews the balance in favor of aberrant deposition and reduced healthy breakdown.

Myofibroblasts deposit large quantities of collagens and other matrix components, forming thick bands of scar tissue where delicate alveolar walls once resided. Collagen fibers that are disordered and stiff eventually disrupting healthy tissue mechanics and culminating in a characteristic disruption of the alveolar spaces and capillaries.

Over time, the remodeling extends beyond the initial sites: adjacent alveoli merge, small airways collapse or dilate, and the lung architecture becomes a patchwork of fibrotic and relatively spared areas (the classic spatial heterogeneity of IPF pathology). The increased stiffness of the lung due to fibrosis leads to a restrictive lung physiology (low compliance, reduced lung volumes) and contributes to hypoxemia (impaired gas exchange). The stiff matrix also feeds back to promote further fibrosis—for example, the abnormal matrix can sequester growth factors (like latent TGF1-β) and activate them, and the stiffness can induce fibroblasts to continue producing collagen. Establishment of a self-sustaining loop results in continuing the progression of the fibrosis even if an initial injury subsides.

Active TGF-β1 binds to TGF-β receptors on target cells, activating SMAD and non-SMAD pathways that drive fibroblast proliferation, transformation into myofibroblasts, and synthesis of collagen and fibronectin. TGF-β also suppresses immune responses and inhibits epithelial regeneration, ensuring that fibrous tissue replaces normal tissue.

TGF-β1 can modulate the expression of key components of the Hedgehog pathway, including Sonic Hedgehog (Shh), independent of Smoothened (Smo), a key signal transducer in the pathway. TGF-β1 also can induce Hedgehog-Gli signaling activation and promote Smad2/3-dependent EMT as has been observed in some cancer cell types.

The hedgehog pathway plays a key role during fetal lung development and lung fibrogenesis. Given that the hedgehog pathway is continuously upregulated in pulmonary fibrosis leading to fibroblast activation and myofibroblast differentiation as described above, well as macrophage activation and polarization, both of which result in progressive lung fibrosis, inhaled hedgehog inhibitors pursuant to the present invention offer a therapeutic option not currently explored clinically. Normal hedgehog pathway signaling regulates repair of the lung epithelium and is typically only transiently expressed. Hedgehog pathway expression is continuously up-regulated in lung fibrosis and plays a key role in remodeling damaged lung epithelium. First, hedgehog pathway signaling controls fibroblast activation and myofibroblast differentiation. Second, hedgehog inhibitor signaling regulates EMT: cross-talk between hedgehog pathway genes with other developmental pathways, growth factors such as TGF-beta and microenvironments that induce fibrogenesis.

In addition, hedgehog pathway signaling down regulates macrophage activation and polarization. All these effects are synergistic in halting or ameliorating lung fibrosis. As a class, inhaled hedgehog pathway inhibitors are treated as Hedgehog inhibitors as active pharmaceutical ingredients, drugs, or compounds that target and block the Hedgehog signaling pathway, which is crucial for embryonic development and tissue regeneration and that play a key role in the regulation of cell growth and differentiation. Abnormal activation of the Hedgehog pathway can lead to various cancers, such as basal cell carcinoma, medulloblastoma, and other tumors. Therefore, Hedgehog inhibitors have been explored primarily as cancer treatments.

Moreover, the physicochemical properties of the resulting aerosol created by the compositions and methods of the present invention are an important part of the therapeutic utility of the present invention because the specially selected formulation design parameters, together with dry powder dispersion by the dry powder inhaler structures as described below yield an aerosol powder cloud that has uniquely advantageous properties for delivery of the active ingredient to a pulmonary compartment that is tailored to the pharmacodynamic absorption of the active pharmaceutical ingredient in the deep lung than about 5 μm. With respect to certain terminology: the term “mg” refers to milligram, the term “μg” refers to microgram. The term “approximately” indicates that a therapeutically effective pharmaceutical dose includes that values slightly outside the cited values, e.g., plus or minus 0.1% to 10%, which are also effective and safe. As used herein, the terms “comprising,” “including,” “such as,” and “for example” are used in their open, non-limiting sense.

The terms “administration” or “administering” and “delivery” or “delivery” refer to a method of giving to a human a dosage of a therapeutic or prophylactic formulation of the inhaled out pathway inhibitor. When a particular dry particle formulation is described as enabling deposition to the “deep lung,” the formulation is tailored to pulmonary administration, or inhalation or pulmonary delivery as a composition or method of delivering to a human a dosage of the therapeutically effective or prophylactic formulation is delivered to the lungs of a human having been diagnosed with the progressive pulmonary fibrotic disorder. The term “actuation” of “actuations” refers to triggering a dry powder delivery device to deliver the designated unit dosage of a drug formulation via the dry powder inhaler to achieve deep lung deposition.

A “carrier” or “excipient” is a compound or material used to facilitate administration of the compound, for example, to increase the solubility of the compound. Solid carriers include, e.g., equivalent such compounds are described in the literature, e.g., in the Merck Index, Merck & Company, Rahway, NJ. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

The terms “D10, D50 and D90” refer to volume-based diameters of particles at the 10th, 50th and 90th percentile. Particle sizes are described herein by reference to the Dv50 value, which is the median particle size for a volume distribution. Thus, half the volume of the particles have diameters of less than the Dv50 value and half the volume of the particles have diameters of greater than the Dv50 value with conventional descriptions of describe particle size distributions. The parameters of Dv10 and Dv90 are also used to characterize a in-industry accepted particle size distribution of a sample such that 10% of the volume of particles have a diameter of less than the Dv10 value. 90% of the volume of the particles have a diameter of less than the Dv90 value. Techniques to measure the Dv50 (and Dv10 and Dv90) values are well known in the art and include laser diffraction.

When referring to a dry powder delivery device, the term “low resistance” refers to a dry powder inhalation device whereby about 100 liters per minute is required to generate the 4 kPa pressure drop required to actuate and disperse dry powder particle population contained therein. The term “medium resistance” refers to a dry powder inhalation device whereby about 85 liters per minute is required to generate the 4 kPa pressure drop required to actuate and disperse the dry powder inhaled hedgehog inhibitor pathway particles described therein. The term “high resistance” refers to a dry powder inhalation device whereby about 60 liters per minute is required to generate the 4 kPa pressure drop required to actuate and disperse the inhaled hedgehog pathway dry powder particle formulations and unit doses described herein.

A “therapeutic effect” relieves, to some extent, one or more of the symptoms associated with progressive pulmonary fibrotic disease and may be defined in terms of is defined as a reduced level or rate of a morphology and lung tissue consistent with progressive pulmonary fibrotic disease The term “prophylactic treatment” refers to treating a patient who is not yet diseased but who is susceptible to, or otherwise at risk of, a particular disease, or who is diseased but whose condition does not worsen while being treated with the pharmaceutical compositions described herein. The term “therapeutic treatment” refers to administering one of the therapeutic treatments described herein in a therapeutically effective amount and directly to deep lung tissue to achieve cellular adsorption at the alveolar membrane.

Two major designs of dry powder inhalers are currently available. One design is the metering device in which a reservoir for the drug is placed within the device and the patient adds a dose of the drug into the inhalation chamber. The second is a factory-metered device in which each individual dose has been manufactured in a separate container. Both systems depend upon the formulation of drug into small particles of mass median diameters from 1 to 5 microns, and usually involve co-formulation with larger excipient particles (typically 100 micron diameter lactose particles). Drug powder is placed into the inhalation chamber (either by device metering or by breakage of a factory-metered dosage) and the inspiratory flow of the patient accelerates the powder out of the device and into the oral cavity. Non-laminar flow characteristics of the powder path cause the excipient-drug aggregates to decompose, and the mass of the large excipient particles causes their impaction at the back of the throat, while the smaller drug particles are deposited deep in the lungs.

Current technology for dry powder inhalers is such that payload limits are around 100 mg of powder. The lack of long-term stability of aztreonam in an aqueous solution due to hydrolysis allows dry powder inhaler technology to become a preferred delivery vehicle for aztreonam dry powder. As noted above, particle size and other particle formulation composition, and physical properties of the inhaled hedgehog pathway inhibitor are tailored to achieve the requisite deep lung deposition to achieve the therapeutic effect as disclosed. If the particle size is larger than 5p then the particles are deposited in upper airways. If the particle size of the aerosol is smaller than 1μ then it does not get deposited in the endobronchial space but continues to be delivered into the alveoli and may get transferred into the systemic blood circulation.

A metered dose inhaler consists of three components: a canister containing the propellant drug suspension, a metering valve designed to deliver accurately metered volumes of the propellant suspension, and an oral adapter which contains a spray orifice from which the metered dose is delivered. In the rest position, the metering chamber of the valve is connected to the drug suspension reservoir via a filling groove or orifice. On depression of the valve this filling groove is sealed and the metering chamber is exposed to atmospheric pressure via the spray orifice in the oral adapter and the valve stem orifice. This rapid pressure reduction leads to flash boiling of the propellant and expulsion of the rapidly expanding mixture from the metering chamber. The liquid/vapor mixture then enters the expansion chamber which is constituted by the internal volume of the valve stem and the oral adapter. The mixture undergoes further expansion before being expelled, under its own pressure, from the spray nozzle. On exit from the spray orifice, the liquid ligaments which are embedded in propellant vapor are torn apart by aerodynamic forces. Typically, at this stage, the droplets are 20 to 30μ in diameter and are moving at the velocity of sound of the two-phase vapor liquid mixture (approximately 30 meters per second). As the cloud of droplets moves away from the spray nozzle, it entrains air from the surroundings and decelerates, while the propellant evaporates through evaporation, the entrained droplets eventually reach their residual diameter.

At this point, the particles/droplets consist of a powdered drug core coated with surfactant. Depending on the concentration and the size of the suspended material the powdered drug core consists of either individual drug particles or aggregates. Currently, meter dose inhaler technology is optimized to deliver masses of 80 to 100 micrograms of drug, with an upper limitation of 1 mg of drug deliverable.

An alternated route of dry powder delivery is by the dry powder inhalers described that can have several different designs although two major designs of dry powder inhalers exist: 1) device-metering designs in which a reservoir of drug is stored within the device and the patient “loads” a dose of the device into the inhalation chamber, and 2) factory-metered devices in which each individual dose has been manufactured in a separate container. Both systems depend upon the formulation of drug into small particles of mass median diameters from 1 to 5 microns, and usually involve co-formulation with large excipient particles (typically 100 micron diameter lactose particles). Drug powder is supplied into the inhalation chamber (either by device metering or by breakage of a factory-metering dosage) and the inspiratory flow of the patient accelerates the powder out of the device and into the oral cavity. Non-laminar flow characteristics of the powder path cause the excipient-drug aggregate to decompose, and the mass of the large excipient particles causes their impaction at the back of the throat, while the inhaler drug particles are deposited deep in the lungs. Current technology for dry powder inhalers is such that payload limits are around 50 mg of powder (of which drug is usually a partial component by mass). Excipients commonly used are lactose, however in the case of aztreonam free base the addition of the amino acids lysine or leucine will lead to better powder formation.

United States Food & Drug Administration (FDA) approved hedgehog inhibitors compounds include:

Vismodegib (Erivedge)—One of the first FDA-approved Hedgehog inhibitors, used to treat advanced basal cell carcinoma.

Sonidegib (Odomzo)—Another FDA-approved inhibitor, used for similar indications as vismodegib.

Glasdegib—Often used in combination with other chemotherapy agents to treat acute myeloid leukemia (AML).

Screening for candidate hedgehog pathway inhibitors within the scope of the present invention is achieved by affirmatively testing for several parameters including three specific parameters as follows:

### 1. Achieving a Plasma Binding of >90%

Measurement of the plasma binding of an inhaled hedgehog pathway inhibitor assesses how much of the inhibitor compound is bound to plasma proteins versus how much is free or unbound. The unbound fraction is the only pharmacologically active form that interacts with the hedgehog pathway proteins. Measurement may be achieved by number of one-month techniques including equilibrium dialysis, using a semipermeable membrane, ultrafiltration, centrifugation, or others to yield a bound and unbound fraction as the concentration of the total drug concentration in serum. Many inhalable hedgehog pathway inhibitors including vismodegib in particular are highly protein-bound to albumin and alpha-1 acid glycoprotein proteins and may require establishment of a concentration range gradient to accurately assess the extent of plasma point.

2. Devising a formulation resulting in a Plasma half life of less than 5 days to exclude compounds that could lead to systemic buildup of the inhaled hedgehog pathway inhibitors and the accordant risk of systemic HHi adverse effects.

3. Achieving a total targeted aerosol dose of less than 5 mg to avoid any higher doses that could cause a cough response to the dry powder particles as described herein.

As noted above, hedgehog pathway inhibitors have the effect of by blocking the interaction between the Hedgehog protein and its receptor, thereby preventing the downstream signaling that can lead to uncontrolled cell growth of myofibroblasts. In considering a candidate formulation for inhaled inhibition of the hedgehog pathway, the following analysis, factors, and calculation can be used both as a basis to include or exclude a candidate hedgehog pathway inhibitor API.

Steady-state levels of a candidate inhaled hedgehog pathway inhibitor are generally achieved after about 4 to 5 half-lives of a candidate API. For comparison, given that the half-life of vismodegib is 3 days, it would take approximately 12 to 15 days to reach steady state.

This calculation assumes the API is being administered in a clinically effective formulation as disclosed herein, at regular intervals, and that the pharmacokinetics of the candidate compound follow first-order kinetics where the rate of drug elimination is proportional to its concentration. To calculate the multiple of the initial drug concentration at steady state (known as the “accumulation factor”), the formula:

Accumulation Factor (AF)=1/(1−e\hat{ }(−ke*τ)) is applied where:
Ke is the elimination rate constant, calculated as ke=0.693/half-life; τ (tau) is the dosing interval, in this case, 1 day.

### Steps:

Calculate ke: For a half-life of 3 days: ke=0.693/3≈0.231 day⁻¹.

Substitute into the formula: With τ=1 day: AF=1/(1−e\hat{ }(−0.231*1))≈1/(1−e\hat{ }(−0.231)).

Evaluate the exponential: e\hat{ }(−0.231)≈0.794.

Finish the calculation such that: AF=1/(1−0.794)=1/0.206≈4.85.

Therefore, at steady state, the candidate API concentration would be about 4.85 times the initial concentration after the first dose. An example of the inhaled vismodegib disclosed herein, the Accumulation Factor remains low enough to avoid systemic adverse effects and illustrates a discrete in the assessment of the candidate API as disclosed here.

However, as noted above, given that steady-state levels in plasma are generally achieved after about 4 to 5 half-lives of a drug. If the half-life of a candidate API drug is 28 days, then it would take approximately 112 to 140 days to reach steady state.

Under such an example, the accumulation factor (AF) can be calculated at steady state using the same formula:

$$AF = 1 / ( 1 - e \hat{} ( - ke * τ ) )$$

Where: ke is the elimination rate constant: ke=0.693/half-life; (tau) is the dosing interval, which is 1 day.

### Steps:

Calculate ke: For a half-life of 28 days: ke=0.693/28≈0.02475 day⁻¹.

Substitute into the formula: With τ=1 day: AF=1/(1−e\hat{ }(−0.02475*1))≈1/(1−e\hat{ }(−0.02475)).

Evaluate the exponential: e\hat{ }(−0.02475)≈0.9756.

Finish the calculation: AF=1/(1−0.9756)=1/0.0244≈41.

In this example, at steady state, the drug concentration will be approximately 41 times the initial drug concentration after the first dose, assuming consistent daily dosing, first-order kinetics, and no other influencing factors. These results lead to exclusion of a candidate API as an inhaled hedgehog pathway inhibitor because the accumulation would lead to a much higher risk of systemic hedgehog adverse effects. Furthermore, in a clinical study, these adverse effects may not occur for five months, increasing the risk of a late-stage clinical failure.

Exemplary inhaled hedgehog inhibitors or modulators useful in the compositions and methods disclosed herein, including API compounds, pre-packaged pharmaceuticals, drug-device combinations, and methods of manufacturing and treatment include the agents listed in Table 1, without limitation.

TABLE 1

Hedgehog Pathway Modulators
Itraconazole
Posaconazole
Arsenic trioxide
Vitamin D3
Saperconazole
Vismodegib (GDC-0449)
Erismodegib/Sonidegib (LDE225)
Taladegib
XL139 (BMS-833923)
Glasdegib (PF-04449913)
Saridegib (IPI-926)
Auranofin
GANT58
GANT61
Robotnikinin
MRT 10
M 25
U 18666A
RU-SKI 43
JK 184
HPI1
Eggmanone
Ciliobrevin A
AZ 12080282
AY 9944
SMANT
SANT-1
SANT-2
PF 5274857
Jervine
IHR1
TAK-441

### Example 1—Clinical Study to Assess the Use of an Oral Hedgehog Inhibitor In Idiopathic Pulmonary Fibrosis

Two clinical studies with oral hedgehog inhibitors were conducted in IPF to demonstrate that inhibition of the hedgehog pathway is a therapeutically effective option to a progressive pulmonary fibrotic disorder albeit with a very high adverse event rate.

In one study, 21 IPF patients were enrolled in a phase 1b open-label trial to receive vismodegib 150 mg one daily plus pirfenidone 801 mg/day three times daily for 24 weeks (3). On average, lung function and shortness of breath improved during the study. This result is unlike the typical decline reported in patients treated with oral pirfenidone alone. Adverse effects were common, all patients reported at least one treatment-emergent adverse event. Most frequent were muscle spasms (n=16 [76%]) dysgeusia (n=13 [62%]), alopecia (n=7 [33%]), weight loss (n=7 [33%]), and decreased appetite (n=6 [29%]). One AE of decreased appetite of life-threatening intensity occurred and was considered by the investigator to be related to vismodegib. Serious AEs were reported in 14.3% of patients; one event of dehydration was considered related to vismodegib. More patients discontinued vismodegib than pirfenidone (42.9% vs. 33.3%, respectively). The common reported AEs are all caused by systemic hedgehog inhibition.

### Example 2—Randomized Placebo-Controlled Multicenter Study to Assess Clinical Effectiveness of a Hedgehog Inhibitor in Progressive Pulmonary Disease

Another study of the oral hedgehog inhibitor taladegib was a randomized, placebo-controlled, multi-center, 12 week study in 41 subjects with mild to moderate IPF. Following treatment, subjects were observed for an additional 6 weeks. At 12 weeks the taladegib group has on average 1.9% improvement in FVC compared to a 1.3% loss in the placebo group (p=0.035). Furthermore, by high resolution CT scan, the taladegib group had decreased lung fibrosis compared to slight increase in the placebo group. Adverse events in the taladegib group were similar to those reported by Prasse: dysgeusia (57%), alopecia (52%) and muscle spasms (43%). The common reported AEs are all caused by systemic hedgehog inhibition.

The company developing oral taladegib is continuing to develop the drug perhaps in the hope that the adverse event profile would be acceptable if clinical efficacy is proven. There are generally three methods of administration of an inhaled drug to the lung. The first is by a nebulizer with a liquid formulation. Vismodegib is insoluble in an aqueous formulation making this an impractical mode of delivery unless the drug is suspended in a colloid formulation. Another drawback to nebulization is that typically takes from 2 to 10 minutes, much less convenient than a single breath with a dry powder or a meter dose inhaler.

The second method of delivery is by metered dose inhaler. This method requires dissolution or suspension of the active agent in a carrier gas, typically hydrofluoroalkane. The largest practical dose is approximately 1 mg, so this could be a potential delivery system but would be limiting if doses greater than 1 mg are needed.

The third method of delivery is by a dry powder inhaler. Large dry powder dosing, greater than 5 mg of active pharmaceutical ingredient (typically blended into excipients 10-20 fold higher in mass) is associated with coughing, so a low dose is required. Dry powders can be prepared by milling or spraying the compound after it is dissolved in an organic solvent.

A fourth method of delivery is heating the compound to create a vapor that is then subsequently inhaled. This requires the API to be thermally stable.

The present disclosure further relates to the development of a dry powder formulation of vismodegib that is 9,000 more effective in lung delivery than the oral drug. This far exceeds by over an order of magnitude the typical advantage that inhaled delivery has over oral and will lead to systemic exposure levels that will eliminate any adverse event due to hedgehog inhibition. The amount needed for efficacy after inhaled delivery is unexpected and surprising.

The basis for this is the published pharmacokinetics of oral vismodegib and estimates of inhaled drug delivery. Vismodegib has the unusual property of saturable oral absorption where one cannot increase plasma concentrations with doses higher than the FDA approved 150 mg daily dose. In a phase I study of 150 mg daily dose of vismodegib, skin biopsies showed that GLI1 expression (a marker of hedgehog inhibition) was down-modulated>2-fold in 25 (73.5%) of 34 patients with locally-advanced or metastatic solid tumors. At steady state the 150 mg daily oral vismodegib dose has a peak plasma concentration of 22.6±10.8 μmol/L. However, it is 98% plasma protein bound so the mean unbound concentration is 0.109±0.058 μmol/L (6). Only unbound drug can freely cross the alveolar epithelial barrier. Thus, a target concentration of approximately 0.109 μmol/L in the epithelial lining fluid should mimic the efficacy results in the Prasse study. The half-life of oral vismodegib is three days, so, with once daily oral therapy, the plasma concentration is likely at a nadir of about 80% of the peak.

The physical delivery of any drug by the inhalation route may be estimated by data generated in the delivery of inhaled pirfenidone. In the Phase 1 study of aerosolized pirfenidone, 24 hour urine analysis confirmed that of the 100 mg dose in the nebulizer 45 mg was delivered to the lung. The epithelial lining fluid concentration in normal volunteers averaged 135 ug/mL. Plasma and urine data confirmed similar delivery in an IPF patient cohort. Since delivery is just a physical process, one can estimate ELF concentrations for 400 μg of vismodegib nominal dose in dry powder format using the Berry R01 inhaler.

The Berry RS01 device has been in use for over 35 years for multiple drugs including patients with interstitial lung disease. To use the device a patient inserts a single capsule into the device at each dosing. The optimum load to the device is 10-30 mg in one capsule, typically the carrier, typically the excipient represents greater than 95% of the mass.

Predicted ELF concentration if 45 mg delivered=135 μg/mL=135,000 μg/L

Molar concentration (MW 348) if 45 mg delivered=135,000 μg/L/348=388 μmol/L

The Berry RO1 delivery device is about 50% in deep lung delivery. Adjusting for 200 pg delivery (50% of 400 pg), 200 ug/45 mg=0.0044 388 μmol/L×0.0044=1.71 μmol/L

Comparing to the unbound concentration of the oral 150 mg dose 1.71 μmol/L/0.109 μmol/L≈16

Therefore, a 400 μg inhaled delivered dose would be predicted to have over 16-fold higher vismodegib levels in ELF than what is achieved by a 150 mg oral dose of vismodegib. This margin allows additional coverage if a patient's inhalation technique is not optimal.

### Example 3—Dry Powder Formulation of Vismodegib in 4 Matrices for Inhaled Delivery to the Deep Lung

A 4% spray dried formulation of vismodegib was developed in four matrixes. The minimal nominal dose (drug+excipients) is 10 mg, which is the 400 ug dose. The maximum in a capsule is 30 mg which would be a 1.2 mg nominal dose with 600 ug delivered.

Four dry powder formulations were developed using spray drying with two being acceptable for human use. Although vismodegib is insoluble in water at physiologic pH's, a 50:50 EtOH: water (wt/wt) solution could dissolve 4% vismodegib. The selected bulking agents were mannitol or trehalose with dispersibility enhancer: L-leucine (˜0.55 wt % solubility limit in 50:50 EtOH: water). Two mannitol formulations and two trehalose formulations with different amounts of L-leucine 10% and 26% were made using a BLD-35 spray dryer. The tested dose was 10 mg of the formulation in a Berry RSO1 high resistance inhaler. Therefore, the nominal dose in the capsule was 400 μg of vismodegib. This device can accommodate up to 30 mg of formulation with 1.2 mg of vismodegib.

All formulations depicted good aerosol performance and reached the target active fine particle distribution of 250 μg of vismodegib delivered to the deep lung. The emission efficiency was also good, evidenced by the range of emitted fraction (84-93%). The formulation with the higher leucine content resulted in slightly higher fine particle distribution, indicating deagglomeration efficiency increases with increasing leucine content. The parameters demonstrated for vismodegib are readily applicable to taladegib, particularly in the quantities and dry particle parameters described herein. The quantities, dosages, excipients, particle size and formulation parameters described in the present indication for an inhaled hedgehog pathway inhibitor for progressive pulmonary disease are also readily applicable to the other methods of treatment described herein and rely on analogous methods of manufacture well known to those of ordinary skill in the field. One skilled in the field can also apply the teachings herein to additional species of inhaled hedgehog pathway inhibitor to create an inhaled composition, treatment modality, or method of manufacture by applying the inclusionary and exclusionary criteria for selecting a candidate hedgehog pathway inhibitor for inhalation, ability to reach the deep lung tissue, and other clinical parameters described herein and that may be extrapolated to other hedgehog inhibitor species.

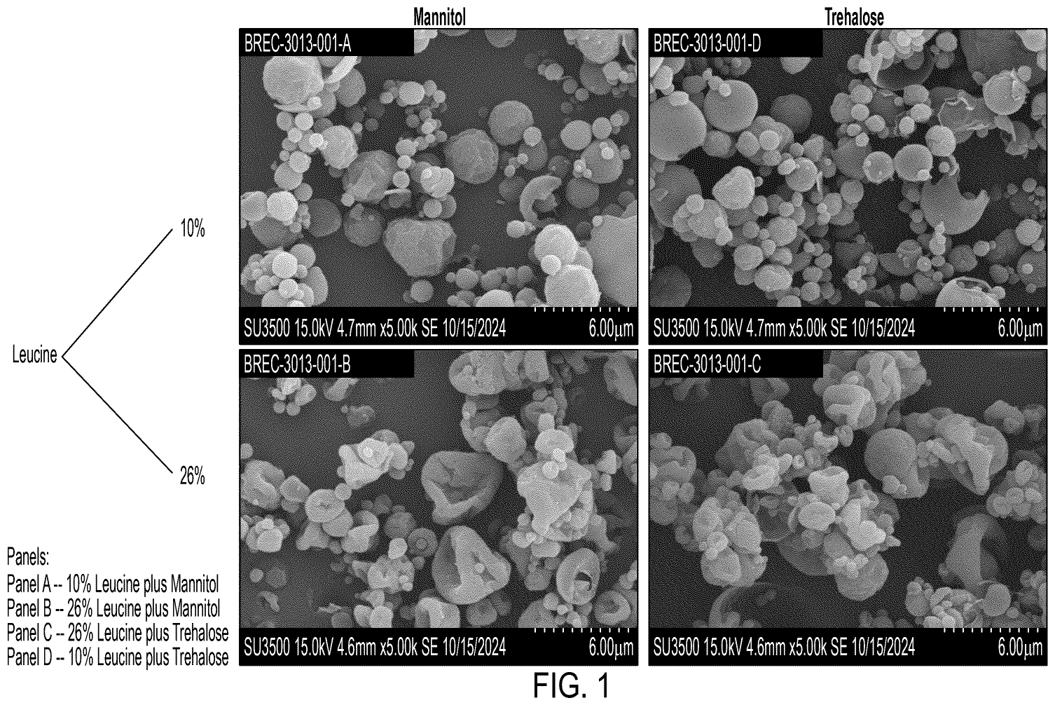

Referring to FIG. 1, collapsed spheres are the desired particle morphology and are indicative of successful particle engineering. FIG. 1 is a microscopic view of four artifacts of successful particle engineering for two different leucine concentrations together with both mannitol or trehalose. Panels B and C achieve the desired property of the majority of the particle population comprising collapsed spheres indicating the requisite leucine surface enrichment consistent with the present invention that yield a particle morphology capable of delivery to the deep lung. A majority of a particle population comprising collapsed spheres was achieved with leucine surface enrichment formulations comprising greater than 10% leucine, and including the 26% leucine enriched formulations as described in FIG. 1 and as high as 50% because Values higher than 50% are not possible due to the limited solubility of leucine. As is apparent from panels B and C of FIG. 1, this morphology required for deep delivery to the lung are achieved, but the 10% leucine formulations were predominantly spherical, suggesting that, although 10% or less leucine formulations may be adequate with other formulations and API, the 10% leucine for vismodegib and taladegib was not enough leucine surface enrichment (see predominantly spherical particles in panels A and D) for this formulation to achieve the desired morphology.

All formulations were within desired potency range (98-102%) post correction for water and solvent content. The formulations with 10% leucine did not have significant leucine crystallization, indicating insufficient leucine surface enrichment. Formulation with trehalose with 26% leucine had the largest leucine crystallites, and therefore more extensive leucine surface enrichment creates a hydrophobic shell which protects against water uptake on stability, and it is a dispersibility enhancer, leading to better aerosol performance.

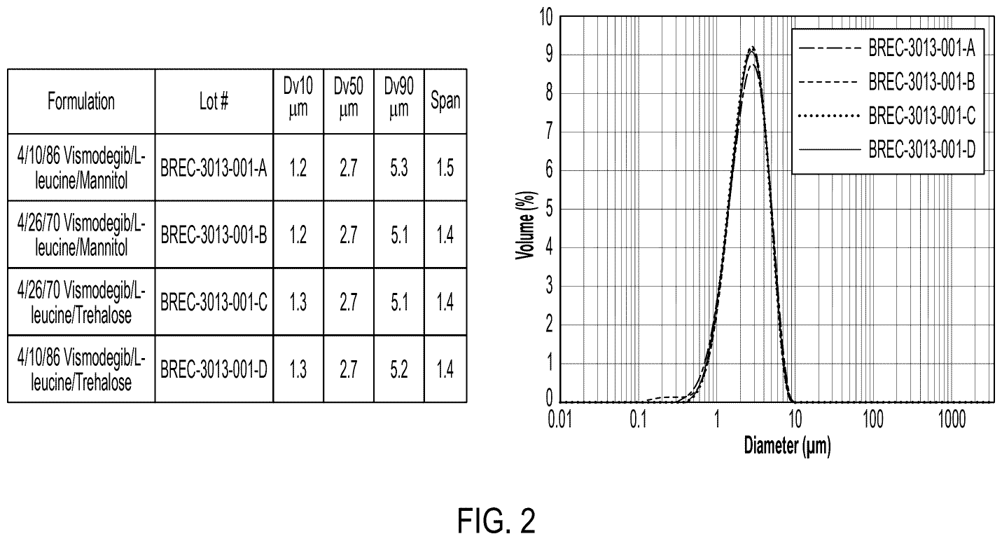

FIG. 2 is a geometrical particle size distribution (GPSD) achieved by the formulations described herein in a preferred range of values validating the manufacturing process parameters disclosed herein and showing a low variable range-maintaining particle size distribution in a desired respirable range for deep lung deposition. Referring to the left panel in FIG. 2, four lots (BREC-3013-001-A, 001-B, 001-C and 001-D, hereinafter “lots 001A-D”) were tested for particle size distribution with vismodegib at 10% and 26% L-leucine surface enrichment with either of 70% or 86% mannitol or trehalose as indicated. Referring to the right panel of FIG. 2, and although, as noted in FIG. 1 the higher levels of L-leucine enrichment, are preferred, each of lots 001-A through 001-D achieve the target and narrow particle size distribution for each formulation. Furthermore, the tight particle size distribution of the spray-dried powder as disclosed and taught herein is also within respirable range parameters and was demonstrated to be feasible pursuant to the methods of manufacture described herein. Specifically, the Dv₁₀ is greater than 1 μm, the Dv₅₀ is greater than 2 μm and less than 3 μm, and the Dv₉₀ is less than 6 μm.

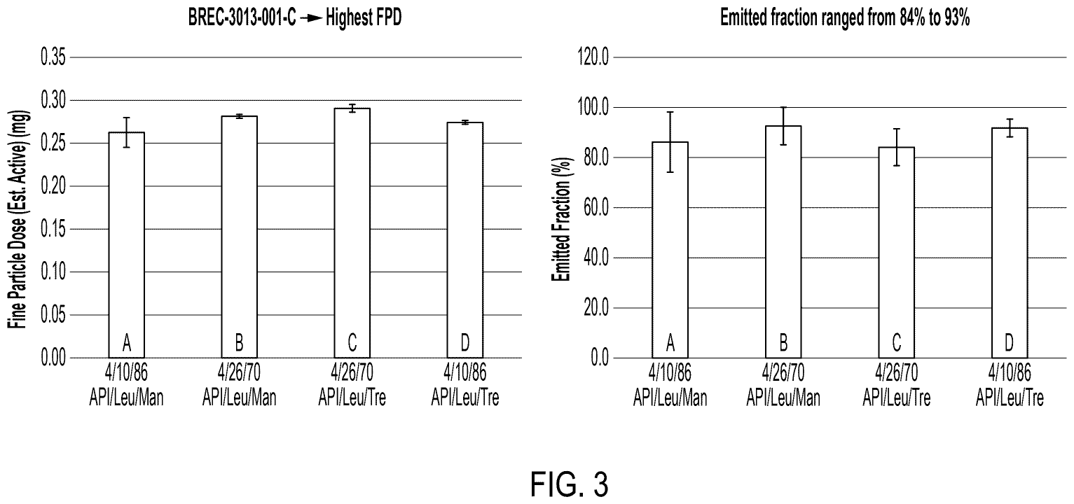

FIG. 3 is fast screening impactor results for four separate engineered dry powder compositions for combinations of inhaled hedgehog pathway inhibitors incorporated into the engineered dry particles, containing two levels of L-leucine, and alternatively mannitol or trehalose. Each formulation in combination of materials that showed the desired parameters necessary for active dose delivery to the deep lung (approximately 250 μg) based on an observation of homogeneity and a consistent distribution of 4% API throughout the dry particle population distribution.

Referring to the left panel of FIG. 3, the lot 001-C shows nominally the highest Fine Particle Dose (FPD) at slightly over 29 mg estimated activity based on 26% L-leucine 4% API and 70% trehalose—although all lots show the ability to distribute as unit dose for deep lung delivery. Turning to the right panel of FIG. 3, Lots 001-A through 001-D all show an high emitted fraction range 84% to 93%. The left panel shows that all the tested materials establish aerosol performance reaching the target fine particle dose of 250 μg vismodegib delivered to the deep lung (left panel) with an emissions frequency also demonstrating and emitted fraction capable of achieving deep lung deposition.

The present disclosure results in a 16-fold higher vismodegib endothelial lining fluid (ELF) level than that achieved with the oral drug which would maintain therapeutic levels for 5 half-lives. Because vismodegib is not water soluble, diffusion across the alveolar capillary membrane will be slow, and, therefore, achieves a relatively long ELF half-life for inhaled vismodegib. Thus, a minimum of once a day dosing is sufficient for efficacy. A measurement of ELF half-life in a sheep model can readily be used to choose an optimal unit dose, or series of clinical doses clinical dose and dosing schedules to tailor the amount and frequency amount and frequency to achieve therapeutic efficacy. Accordingly, any individual half-life for an inhalable hedgehog pathway inhibitor does not give target coverage for 24 hours, the number of administrations per day can be increased typically within a maximum of four doses per day.

The adverse effects of oral vismodegib are caused by systemic exposure and the 150 mg dose has 25% systemic absorption, approximately 37 mg. Even in the event of a short half-life requiring more frequent dosing at higher dose levels, systemic exposure remains low as compared to oral dosing. For example, six administrations a day of a 30 mg capsule (nominal dose 1.2 mg, delivered dose of 600 pg) would give total systemic exposure of 3.6 mg which is less than 10% of the oral dose exposure and unlikely to cause hedgehog pathway-related adverse effects. Similar advantages of inhalation can be predicted for taladegib.

### Example 4—Dry Powder Formulation for Delivery of Taladegib to the Deep Lung

At steady state the 200 mg daily oral taladegib dose has a mean concentration of about 0.8 μmol/L. Only unbound drug can freely cross the alveolar epithelial barrier and taladegib is 95% bound so the unbound taladegib concentration would be approximately 0.04 μmol/L.

Again, since delivery is just a physical process, one can estimate ELF concentrations for 200 μg of taladegib delivered as noted above based on pirfenidone data.

Predicted ELF concentration if 45 mg delivered=135 μg/mL=135,000 μg/L

Molar concentration (MW 512) if 45 mg delivered=135,000 μg/L/512=264 μmol/L

Adjusting for 200 pg delivery 200pg/45 mg=0.0044 264 μmol/L×0.0044=1.16 μmol/L

Comparing to the unbound concentration of the oral 200 mg dose 1.61 μmol/L/0.04 μmol/L≈29

Therefore, a 200 μg inhaled delivered taladegib dose would be predicted to have over a 29-fold higher levels in ELF than what is achieved by a 200 mg oral taladegib dose. This would be about 6 half-lives. Depending on the half-life in the epithelial lining fluid, one to six inhalations a day would provide adequate inhibition of hedgehog receptors. At the highest dose frequency (6 inhalations/day), the total delivered dose would approximate 3.6 mg. The effective oral dose of taladegib is 200 mg, with approximately 30% absorption. Therefore, systemic absorption would be less than 10% of the oral dose and would likely result in no systemic adverse effects from hedgehog inhibition.

In some embodiments of the present disclosure, the lung dose of an inhaled powder using a high-resistance dry powder inhaler (DPI) device, increases as the primary particle size of the powder decreases. This observation facilitates balancing of the primary particle size with the as delivered lung dose and can be used to enable designing optimized formulations of inhaled hedgehog pathway inhibitors.

In some embodiments of the present disclosure, the principle of increased lung dose with decreased particle size is generally applicable. However, the specific DPI device may influence the extent of this effect due to differences in airflow resistance and dispersion mechanisms. Particle size in the range of 2-5 μm is ideal, balancing deposition in both the small airways and alveoli. Particles less than 1 μm are often exhaled, leading to inefficient drug delivery. A 4 μm particle has an 8-fold higher mass than a 2 μm particle, impacting the total lung dose delivered. Porous particles with lower density can deposit deeper in the lung, but no FDA-approved drugs currently use this technology.

### Example 5—Effect of Particle Size of Inhaled Hedgehog Inhibitors on Lung Deposition to Treat Progressive Pulmonary Disorders

A cascade impactor study was performed to measure aerosol performance for Hedgehog inhibitor dry powder formulations. Three different particle size distributions were tested:
2.7 μm MMAD
3.6 μm MMAD
5.4 μm MMAD

### Results:

The 2.7 and 3.6 μm formulations delivered approximately double the lung dose compared to the 5.4 μm formulation. The 5.4 μm formulation showed a sharp drop in deep lung delivery, aligning with established particle deposition models. These results confirm that an optimal range of 3-4 μm balances deep lung penetration and adequate mass deposition.

### Example 6—Clinical Implications of Small Airway Deposition

Small airway deposition of Hedgehog inhibitors could be beneficial in treating interstitial pulmonary fibrosis (IPF) and other lung diseases where fibrotic processes are active. Delivering the drug in 2-5 μm size range ensures optimal small airway and alveolar deposition, balancing therapeutic effect and safety. Unlike some other drug products such as aerosolized pentamidine, which required 1-2 μm particles to avoid bronchospasm, the Hedgehog inhibitor formulations do not have known bronchospastic effects, making small airway delivery a viable approach.

Cascade impactor-measured aerosol performance refers to in vitro particle size analysis used to predict in vivo deposition. As particle size decreases, cascade impactor results show an increase in predicted lung dose. This method ensures that formulations are optimized for deep lung deposition before human trials. In conclusion, target particle size of 3-4 μm ensures deep lung penetration while maintaining adequate drug mass. Cascade impactor data serves as an in vitro predictor of in vivo lung deposition, guiding formulation development. Because of the lung periphery, i.e. deep lung tissue is a key target for hedgehog pathway, precise control over particle size and morphology in the majority of the particle population is provided in detail herein along with the companion inhalation device performance characteristics to yield maximum therapeutic effect.

This invention provides an optimized dry powder hedgehog pathway inhibitor formulation that balances deep lung delivery, drug mass efficiency, and therapeutic efficacy, ensuring effective treatment. A method to treat pulmonary fibrosis with an inhaled hedgehog inhibitor with unit doses or collective or repeat dosing 1-4 times a day, one to seven times a week, once daily, every fortnight, or once a month.

Specific uses for medical treatment or the first use of the dry powder hedgehog pathway inhibitors disclosed herein include providing hedgehog inhibitor vismodegib dose of up to 1.2 g and specifically within the range of approximately 400 mcg to approximately 1.2 gm of active drug in a dry powder.

The specific uses for medical treatment for the first medical use of the dry powder hedgehog pathway inhibitor taladegib includes doses up to 3.6 mg and preferably in the range of approximately dose of 200 mcg to 3.6 mg of active drug in a dry powder.

A method to prevent radiation fibrosis and pneumonitis with an inhaled hedgehog inhibitor with unit doses or collective or repeat dosing 1-4 times a day, one to seven times a week, once daily, every fortnight, or once a month prior to starting radiation therapy and with the hedgehog inhibitor vismodegib unit dose or repeated dosing includes less than 1.2 gm per dose or between 400 mcg to 1.2 gm of active drug in a dry powder and for hedgehog inhibitor taladegib a unit dose or total dosage less than 3.6 mg of preferably between of 200 mcg to 3.6 mg of active drug in a dry powder inhaler.

A method to treat radiation pneumonitis and fibrosis with the administration or first use with an inhaled hedgehog inhibitor with dosing 1-4 times a day, one to seven times a week, once daily, every fortnight, once a month starting concurrently, during, or after radiation achieved immeasurable anti-fibrotic effect when tracked along with radiation treatment in comparison to radiation treatment alone. As above, the preferred dosage of the hedgehog inhibitor vismodegib dose is less than 1.2 mg and preferably within the range of approximately 400 mcg to approximately 1.2 gm of active drug in a dry powder. The preferred dose of the hedgehog inhibitor taladegib in this indication is similarly at a dose of up to 3.6 mg and preferably within the dose of approximately 200 mcg and approximately 3.6 mg of active drug in a dry powder.

A method to ameliorate lung damage from environmental toxic chemical exposures, including burn pits with an inhaled hedgehog inhibitor ad/or high-altitude radiation exposure or a first use of an inhaled dry powder particulate hedgehog pathway inhibitor includes with dosing 1-4 times a day, one to seven times a week, once daily, every fortnight, once a month. For example, the same dosages of the inhibitor vismodegib would apply, up to 1.2 mg and preferably between 400 mcg to 1.2 gm of active drug in a dry powder particulate format administered with the devices and according to the instructions provided above. Similarly, the method using the hedgehog inhibitor taladegib dose comprises a dose less than 3.6 mg and preferably substantially between 200 mcg to 3.6 mg of active drug in a dry powder.

A method to enhance lung cancer response treating prior to or in conjunction with radiation treatment or chemotherapy exposure together with an inhaled hedgehog pathway inhibitor as described herein with dosing 1-4 times a day, one to seven times a week, once daily, every fortnight, once a month. The hedgehog inhibitor vismodegib at a dose of 400 mcg to 1.2 gm of active drug in a dry powder and the hedgehog inhibitor taladegib dose of 200 to 3.6 mg of active drug in a dry powder

A method to allow higher levels of radiation exposure treating prior to or in conjunction with radiation therapy for the treatment of cancers using radiation to the chest wall with an inhaled hedgehog inhibitor with dosing 1-4 times a day, one to seven times a week, once daily, every fortnight, once a month. The hedgehog inhibitor vismodegib dose of 400 mcg to 1.2 gm of active drug in a dry powder. The hedgehog inhibitor taladegib dose of 200 to 3.6 mg of active drug in a dry powder for treatment of respiratory diseases.

Accordingly, consistent with the above disclosure, the present invention includes each aspect as follows:

A unit dose of an inhaled hedgehog pathway inhibitor having therapeutic effect in a pulmonary progressive fibrotic disorder comprising:
dry powder particles comprising the hedgehog pathway inhibitor and sized to achieve the therapeutic effect in deep lung tissue by cellular adsorption at a surface of an alveolar membrane having histological characteristics of the pulmonary progressive fibrotic disorder, including where the inhaled hedgehog pathway inhibitor is inhaled vismodegib or taladegib and also has, further comprising L-Leucine as an excipient and may further comprise mannitol or and trehalose and combinations thereof as excipients.

The particle characteristics include where the population of the dry powder particles are comprised of a majority of collapsed spheres and optionally where the L-leucine concentration is between 10 and 50% of the dry powder particle population and has a geometric particle size distribution smaller than 5 μm, and optionally between 3-4 μm and/or were in the unit dose comprises less than 5 milligrams of active pharmaceutical ingredient, for example, between approximately 400 μg and 1.2 mg 400 micrograms of vismodegib or between approximately 200 μg to 3.6 mg of taladegib.

For implementation in the drug-device aspect of the present invention, the dry particle population of the hedgehog pathway inhibitor is disposed in a dry powder inhalation device exhibiting an emitted fraction efficiency of at least 84% and where the collapsed spheres are fragmented particles having a partial crystalline configuration. The device preferably has a compartment containing the unit dose of the hedgehog pathway inhibitor that is activated by a patient treated for the progressive pulmonary fibrotic disorder, in response to a histological examination confirming the disease, and containing the single unit dose in a sealed compartment with an actuator to facilitate delivery of the single unit dose to achieve a therapeutic effect. The combination of the drug-device may include a single dose of the hedgehog pathway inhibitor between approximately 400 μg and 1.2 mg 400 μg of vismodegib or between approximately 200 μg to 3.6 mg of taladegib; the device may have an indicator displaying that the sealed compartment contains a daily dose in condition for delivery to the patient, and preferably has an emitted fraction efficiency of at least 84%.

The particle parameters of the population of particles of the hedgehog pathway inhibitor may be characterized by measuring a particle size distribution has a DVD50 of between 1.2 and 5.3 μm, a DV10 greater than 1 μm, and/or the particle size distribution having a DV90 less than 6 μm.

The method for manufacturing an inhaled dry powder formulation of a hedgehog pathway inhibitor, may include dissolving a hedgehog pathway inhibitor in a solvent comprising ethanol and water, adding an excipient selected from a group consisting of mannitol, trehalose, and L-leucine, and drying the mixture to form a dry powder formulation with a majority of the population of particles of the inhaled dry powder formulation comprising a collapsed sphere morphology for improved aerosol performance. The method of manufacture may further comprise encapsulating the dry powder formulation into a compartment of the dry powder inhaler having actuation means operable by a patient having histological confirmation of the progressive pulmonary disease.

Preferably, the method of manufacture produces a the dry powder formulation exhibiting a potency range between 98% to 102%, a median particle size between 1 and 5 μm to facilitate deposition in the deep lung to lead to a therapeutic effect by cellular absorption at the alveolar membrane.

The dry particle population resulting from the above methods of manufacture may include vismodegib or taladegib as the active ingredient and was manufactured by the drying a solution comprising the hedgehog pathway inhibitor in the ethanol-water mixture together with the excipients including in one example mannitol, trehalose, and L-leucine and combinations thereof. The L-leucine excipient inclusion step typically requires adding greater than 10% but less than 50% L-leucine by weight to enhance dispersibility and lung deposition efficiency.

As part of the method of manufacture, the step of encapsulating the dry powder formulation is comprised of disposing the dry powder formulation in a compartment of the dry powder inhaler containing a unit dose of approximately 400 micrograms (μg) to 1.2 milligram (mg) or a unit vismodegib between approximately 200 μg to 3.6 of taladegib, and may further comprise in-process testing to confirm that the L-leucine concentration in the particle population is greater than 10%.

The invention also includes methods for treating pulmonary fibrosis in a patient in need thereof by providing a dry powder inhalation device containing a unit dose having a predetermined quantity of inhaled dry powder particles of a hedgehog pathway inhibitor; and administering the predetermined quantity of the inhaled dry powder particles of the to the deep lung for adsorption at the alveolar membrane, and for example where the hedgehog pathway inhibitor is vismodegib or taladegib and combinations thereof.

The administering step of the method for treatment may comprise inhaling the unit dose from a dry powder inhaler (DPI) device, such as a Berry RS01 device and an equivalent high, medium, or low-resistance inhaler.

Consistent with the above compositions and methods of manufacture, the inhaled dry powder particles have a L-leucine content greater than 10% and an overall range between 10% and 50% by weight. As an advantage of these formulations, the administering step of the inhaled dry powder particles achieves a deep lung deposition efficiency of at least 33% and achieves an epithelial lining fluid (ELF) concentration at least 16 times higher than the concentration achieved by equivalent 200 milligram (mg) oral dose of taladegib and achieves an epithelial lining fluid (ELF) concentration of at least 29 times higher than the concentration achieved by an equivalent 200 milligram (mg) oral dose of taladegib and/or the administering step of the inhaled dry powder particles comprises a dose between 400 micrograms (pg) to 1.2 milligram (mg) of vismodegib or between 200 μg to 3.6 mg of taladegib.

The administering step of the first use or second use or treatment method comprises delivery of the inhaled dry powder particles at an emitted fraction efficiency of at least 84% and a fine particle distribution that ensures deposition of the dry powder formulation in the deep lung and has a fine particle distribution is between 1 and 5 μm, and optionally between 3 and 4 μm. The administering step may also comprise delivering the unit dose between 400 micrograms (μg) to 1.2 milligram (mg) of vismodegib as a single capsule or up to 2.4 mg as two consecutive capsule inhalations or as a single or 2 consecutive capsule inhalations of taladegib between 200 μg to 3.6 mg.

As used herein and in the appended claims, the singular forms “a”, “an”, and “the” include plural referents unless the context clearly dictates otherwise.

Where a range of values is provided, each intervening value, to the tenth of the unit of the lower limit unless the context clearly dictates otherwise, between the upper and lower limit of that range and any other stated or intervening value in that stated range, is encompassed within the disclosure and can be claimed as a sole value or as a smaller range. Where the stated range includes one or both of the limits, ranges excluding either or both of those included limits are also included in the disclosure.

Where a discrete value or range of values is provided, that value or range of values may be claimed more broadly than as a discrete number or range of numbers, unless indicated otherwise. For example, each value or range of values provided herein may be claimed as an approximation and this paragraph serves as antecedent basis and written support for the introduction of claims, at any time, that recite each such value or range of values as “approximately” that value, “approximately” that range of values, “about” that value, and/or “about” that range of values. Conversely, if a value or range of values is stated as an approximation or generalization, e.g., approximately X or about X, then that value or range of values can be claimed discretely without using such a broadening term.

However, in no way should this specification be interpreted as implying that the subject matter disclosed herein is limited to a particular value or range of values absent explicit recitation of that value or range of values in the claims. Values and ranges of values are provided herein merely as examples.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that the feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

What is claimed is:

1. A unit dose of an inhaled hedgehog pathway inhibitor having a therapeutic effect in a pulmonary progressive fibrotic disorder comprising:
a population of dry powder particles comprising:
a majority of the population of the particles are collapsed spheres incorporating vismodegib or taladegib with an L-Leucine excipient and sized for deposition in the deep lung to achieve the therapeutic effect by cellular adsorption at a surface of an alveolar membrane having histological characteristics of the pulmonary progressive fibrotic disorder.

2. The unit dose of claim 1, wherein dose of vismodegib is less than 1.2 mg.

3. The unit dose of claim 1, wherein the dose of taladegib whom is less than 3.6 mg.

4. The unit dose of claim 1, wherein the L-Leucine excipient is greater than 10% and less than 50% of the unit dose of the dry powder particles.

5. The unit dose of claim 4, wherein the L-Leucine excipient is combined with an excipient selected from the group consisting of mannitol and trehalose and combinations thereof.

6. The unit dose of claim 1, wherein the inhaled hedgehog inhibitor is vismodegib in the unit dose is less than 5 mg per day.

7. The unit dose of claim 1, wherein the population of the dry powder particles has a geometric particle size distribution smaller than 5 μm.

8. The unit dose of claim 7, wherein the population of the dry powder particles has a geometric particle size distribution between 3-4 μm.

9. The unit dose of claim 1, wherein the unit dose contains than 5 milligrams of hedgehog pathway inhibitor as the active pharmaceutical ingredient.

10. The unit dose of claim 1, wherein the unit dose is between approximately 400 μg and 1.2 mg 400 micrograms of vismodegib or between approximately 200 μg to 3.6 mg of taladegib.

11. The unit dose of claim 1, wherein the dry particle population is disposed in a dry powder inhalation device exhibiting an emitted fraction efficiency of at least 84%.

12. The unit dose of claim 1, wherein the wherein the collapsed spheres are fragmented particles having a partial crystalline configuration.

13. The unit dose of claim 1, combined with a dry powder delivery device comprising: a compartment activated by a patient and containing the single unit dose in a sealed compartment with an actuator to facilitate delivery of the single unit dose.

14. The unit dose and dry powder delivery device combination of claim 13, wherein the single dose is between approximately 400 μg and 1.2 mg 400 μg of vismodegib or between approximately 200 μg to 3.6 mg of taladegib.

15. The unit dose and dry powder delivery device combination of claim 13, having an indicator displaying that the compartment contains a single dose in condition for delivery to the patient.

16. The unit dose and dry powder delivery device of claim 13, wherein the device has an emitted fraction efficiency of at least 84%.

17. The unit dose of claim 1, wherein the particle size distribution has a DVD₅₀ of between 1.2 and 5.3 μm.

18. The unit dose of claim 1, wherein the particle size distribution has a DV₁₀ greater than 1 μm.

19. The unit dose of claim 1, wherein the particle size distribution has a DV₉₀ less than 6 μm.
